(19) United States
(12) Patent Application Publication
(10) Pub. No.: US 2012/0283614 A1
(43) Pub. Date: Nov. 8, 2012

(54) Band-aid tm type

(76) Inventor: Todd Allen Bland, Omaha, NE (US)

(21) Appl. No.: 13/068,040

(22) Filed: May 2, 2011

(51) Int. Cl. A61F 13/02

(52) U.S. Cl. 602/48

(57) ABSTRACT

A method and system for using a newly invented band-aid™ type of bandage utilizing a small Gel-Pouch beneath the gauze. When the vinyl or equivalent adhesive's bottom pull-tab is removed, an antiseptic gel, Aloe Vera, or equivalents or prescription medication prescribed according to FDA rules and regulations pertaining to such products, such as for certain wounds of a more serious nature; is removed the gel oozes out through the holes from the gel pouch and onto the gauze providing a cool and soothing relief almost immediately.

It also adds healing power and provides a non-stick surface on the gauze pad. This method is not just intended to be a great band-Aid™ type bandage but can come in larger packages for burn victims. A transparent cover encases each bandage to prevent any medicine leakage and also makes it float and waterproof if gotten wet by any means.

### CROSS REFERENCED RELATED APPLICATIONS

This application claims priority of provisional Patent Application 61/275,709 Filing date Apr. 23, 2010. It was converted to a Provisional Application and additionally is assigned the application Ser. No. 12/799,429 Filing date Apr. 23, 2010 due to the Provisional conversion fee.

N/A Federally sponsored research or development.

The specification of the above referenced patent is incorporated by reference in it's entirety.

### BACKGROUND

Overtime Band-Aid™ type bandages have had a dry or dry antiseptic coating infused into the gauze. The drawback is when a person with a wound places dry bandages on it, sweating between the gauze and the wound naturally occurs to a degree, depending on the severity of the said wound. Making it not as comfortable or as effective fighting infection.

The new invention consists of a well known prior art Band-Aid™ type bandage. FIG. 1 (2) Its is a flat piece of latex with a newly invented flat pouch filled with an antiseptic gel, Aloe-Vera or a prescription medicine as prescribed by a US Doctor according to the FDA'S rules and regulations permitted by law and or equivalent. The size of the said bandage is not limited to the size of a band-Aid™ and can be much larger to treat burn victims.

FIG. 2 (4) A thin layer of vinyl, latex or equivalent is adhered to the top of the pouch, The pouch is filled with antiseptic gel, Aloe-Vera or a prescription medication or equivalent.

The pull tab sticks out of one of the ends of the gauze as seen in FIG. 1 (4). The gauze ends are not adhered to the bandage for the purpose of allowing the pull tab and the adhesive thin layer covering the gel pouch to be removed.

The gauze lies on top of the thin adhesive layer with pull tab that is adhered to the gel pouch. FIG. 2 (3).

The wax type paper or equivalent FIG. 4 (6) protects the bandage and the outer transparent plastic cover FIG. (5) helps keep germs out and prevents any spillage of gel, furthermore the plastic cover will allow the bandages to float incase of the bandages were dropped in water or otherwise.

### SUMMARY OF THE INVENTION

There is a need for an antiseptic gel type Band-Aid™ instead of the well known dry antiseptic types of bandages and other dry bandages. For instance a person burns their finger, the present invention simply has a pull tab that when pulled removes an adhered layer which protects the gel pouch. The gel pouch contains antiseptic gel, Aloe Vera or medication requiring a prescription.

The gel pouch then oozes the antiseptic gel or medication to the gauze pad immediately providing soothing pain relief. Rather then a dry bandage laying on top of the wound waiting for the antiseptic gauze to moisten.

### BRIEF DESCRIPTION OF DRAWINGS

FIG. 1 (1) Illustrates a method for constructing a Band-Aid™ Type bandage.

FIG. 1 (1) depicts the top view of the bandage, (6) shows the air holes in the latex or equivalent; (for people allergic to latex). FIG. 1 (2) illustrates the antiseptic gel pouch and the holes that allow the medicine to ooze onto the gauze pad which is above the antiseptic gel pouch.

FIG. 2 (2) Illustrates the antiseptic gel pouch, Aloe-Vera or medicine requiring a prescription.

FIG. 2 (3) Illustrates the gauze pad.

FIG. 2 (4) Illustrates the pull tab which when removed releases the said antiseptic gel onto the gauze pad.

FIG. 2 (5) Illustrates the pull tab.

FIG. 2 (6) Illustrates the air holes in the latex or non-latex adhesive portions.

FIG. 3 (2) Illustrates the side view of the adhesive layer which protects the gel pouch containing the antiseptic gel.

FIG. 3 (4) Illustrates side view of the pull tab for the thin adhesive layer covering the gel pouch.

FIG. 3 (3) Illustrates the side view of thee gauze pad.

FIG. 3 (5) Illustrates the side view of the pull tab.

FIG. 4 (6) Illustrates the front view of the bandage's interior package.

FIG. 4 (7) Illustrates the front view of the interiors bandage adhesive tab that pulls apart the bandages package.

FIG. 4 (8) Illustrates the outer bandages package made of wax type paper or equivalent.

FIG. 5 (9) Illustrates the two perforations on the transparent cover which when pulled opens the cover which protects the original bandage package.

FIG. 5 (10) Illustrates transparent cover made of plastic or equivalent

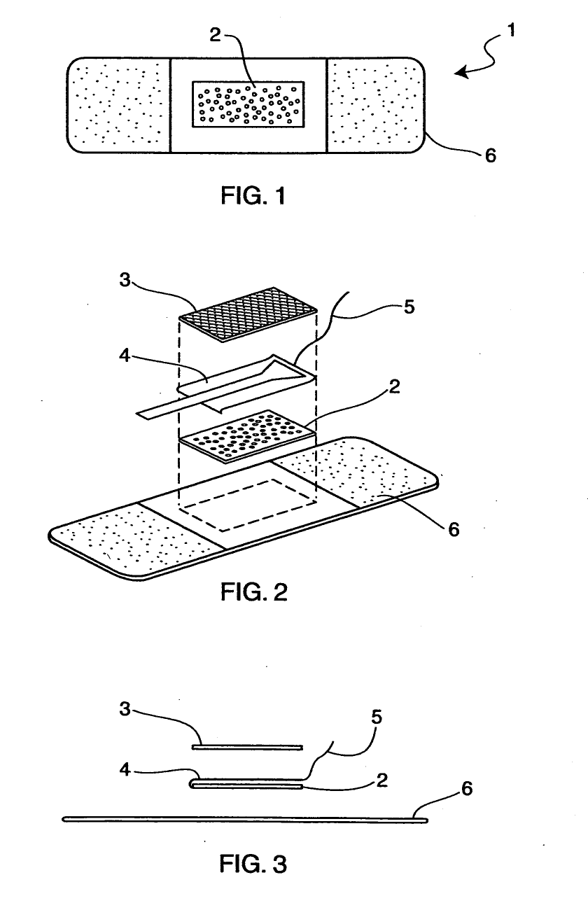

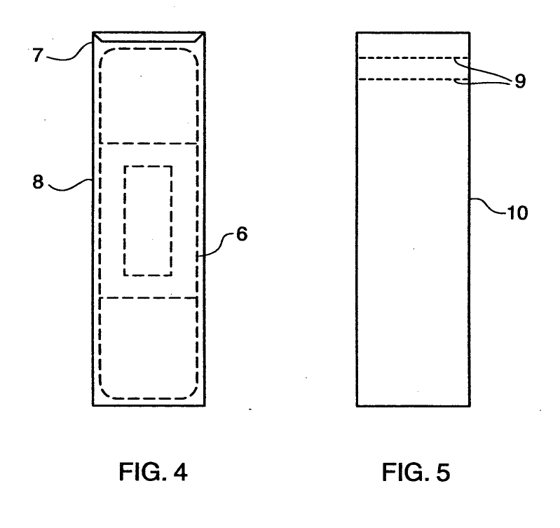

### DETAILED DESCRIPTION OF INVENTION

The newly created Band-Aid™ type bandage which utilizes a flat but small pouch filled with antiseptic gel, Aloe-Vera or

For more serious wounds a prescription medication prescribed by a Certified Physician could be used in the gel pouch, according to the rules of the FDA. Some of the more serious disease could be diabetes, Aid's and other skin disorders, which can limit the dry antiseptic Band-Aid™ substantially.

If a band-aid type “Gel Pouch” requires a prescription due to the medicine in the bandages for a more serious disease such as diabetes, aid's or other skin disorders including burn victims that require a prescription for the “Gel” used in the bandage and can be varied according to the skin disorder including burn victims that will require a must larger “Gel Pouch” bandage. This patent is not intended to limit the newly invented Gel Pouch” to a small band-aid type but also a much larger version capable of handling large burn victims of Adults.

To use simply pull off the transparent cover which has two perforations on the top of the said bandage. This will allow the bandage to float if near the water or if capsized in a boat or prevent gel from leaking from one bandage to the next.

Then the person simply tears apart the band-aid type bandage package that protects the bandage.

The package that protects the bandage is adhered by adhesive on the left and right sides of the said bandage

Preferably the final step is take a hold of the pull tab with your thumb and index finger and pull.

By pulling the bandages tab and releasing the antiseptic gel or equivalent.

After the pulling of the tab, the gel inside of the pouch moves onto the gauze very quickly, allowing almost immediate pain relief and begins the healing process promptly.

What is claimed is as follows:

1. A new bandage called Band-Aid™ type contains a gel pouch containing Antiseptic, Aloe Vera, or prescription medications prescribed by a Doctor.

2. The above mentioned bandage type is not intended to limit he size of the Bandage to a standard Band-Aid™ and is meant to be made large enough to cover large wounds, such as burn victims.

3. A vinyl strip or adhesive layer or equivalent adheres to the gel pouch, protecting the gel until the pull tab is removed.

4-7. (canceled)

8. As set forth in claim 1, the gel pouch once the protective adhesive layer is removed the gel or medicine then saturates the gauze.

9. As set forth in claim 1 the medicine used in the gel pouch may require a prescription due to diseases of a more serious nature, such as diabetes, skin disorders or burn victims.

10. As set forth in claim 3 the top and bottom of the gauze are open to allow the pull tab to be removed. The pull tab is connected to the protective layer protecting the gel in the pouch.

11. As set forth in claim 2 the wax paper or equivalent covers the bandage type bandage and prevents any possible leaking of gel.

12. As set forth in claim 2 the waxed paper or equivalent package is also protected by a transparent cover with one or more perforations at the top of said transparent cover for easy removal.
